(19) United States
(12) Patent Application Publication
(10) Pub. No.: US 2003/0091613 A1
(43) Pub. Date: May 15, 2003

(54) Gelled nutritional composition and process

(76) Inventors: Normanella T. DeWille, Columbus, OH (US); Terrence B. Mazer, Reynoldsburg, OH (US); Jeffery W. Liebrecht, Columbus, OH (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 10/010,465

(22) Filed: Nov. 8, 2001

(51) Int. Cl. A61K 31/70; A61K 47/00

(52) U.S. Cl. 424/439; 514/23

(57) ABSTRACT

A nutritional supplement in the form of a gelled nutritional composition. The composition contains gelled whey protein, a carbohydrate source, minerals, and vitamins. The composition may have a viscosity greater than about 2000 cp, more typically greater than about 5000 cp and an energy density greater than about 600 kcal/liter. The composition is suitable for dysphagia patients, children, and athletes.

### FIELD OF THE INVENTION

This invention relates to a gelled nutritional composition which may provide a nutritional supplement; for example for dysphagia patients and children. The invention also relates to a process for producing the gelled nutritional composition and to a method for providing nutrition to a dysphagic patient or post operatively to a patient recovering from surgery.

### BACKGROUND OF THE INVENTION

Many people do not take in sufficient nutrients for a nutritionally complete diet. In order to assist these people, nutritional supplements have been developed. Nutritional supplements are not intended to provide all the nutrients necessary for a nutritionally complete diet; instead they are intended to supplement the diet such that it becomes more nutritionally complete.

There are many targets for nutritional supplements; for example children, the elderly and patients suffering from dysphagia. Dysphagia, refers to difficulty in swallowing and occurs in all age groups. It is especially prevalent amongst the elderly. Typical symptoms include drooling, a feeling that food is sticking in the throat, discomfort in the chest and throat, a feeling of a lump in the throat, and coughing or choking caused by food not passing easily to the stomach and being sucked into the lungs.

Dysphagia may be caused by many factors. For example, simple causes may be poor teeth, ill-fitting dentures, influenza, or gastroesophageal reflux. Other more serious causes include strokes, progressive neurological disorders, the presence of tracheotomy tubes, paralyzed vocal cords, tumors or surgery to the head, neck or esophageal areas.

Ordinarily, for most people, the condition clears up itself reasonably rapidly or may be easily treated by having appropriate dental work done. However, for more serious cases, the condition may not be easily remedied. In these cases, the patient often suffers weight loss and other complications of inadequate nutrition and liquid intake.

It is generally found that patients suffering from dysphagia are able to better swallow more viscous foods and drinks. Therefore it has been suggested to add viscosity increasing agents to drinks and liquid foods to assist dysphagia patients with swallowing. For example, Japanese patent application 5-38262 describes an agent which is made up of alpha-lized starch and dextrin. The agent may be added to liquid foods to thicken them without changing the color, taste and flavor of the liquid foods. Further, soluble coffee powders which provide thickened coffee beverages are available on the market under the trade name RESOURCE (Novartis Nutrition). These powders contain modified starches to provide the coffee beverage with a “honey-like” consistency.

However, there is still a need for nutritional compositions which may be more readily consumed by dysphagia patients and are of high nutritional value.

Further, for patients in general who require nutritional supplementation, taste fatigue is a common problem; especially among children and the elderly. Nutritional supplements with the new and interesting organoleptic properties and appearances are constantly needed to deal with the problem. In particular, nutritional supplements which do not have a milky appearance and taste are extremely desirable. In order to meet this need, nutritional supplements in the form of fruit juices are described in European patent application 0486425 and U.S. Pat. No. 5,641,531. However, these products are not ideally suited to dysphagia patients. Further, nutritional supplements with other organoleptic properties and appearances are needed.

Therefore an object of this invention is to provide a nutritional composition which is suitable for dysphagia patients and which has a gelled consistency.

### SUMMARY OF THE INVENTION

Accordingly, in one aspect, this invention provides a gelled nutritional composition containing a protein source, which includes gelled whey protein; a carbohydrate source; minerals; and vitamins.

The gelled nutritional composition is in one embodiment, a clear gel. Further, the gelled nutritional composition preferably provides about 30% to about 100% of the recommended daily allowance of vitamins and minerals per 1000 calories. The energy density is preferably about 500 kcal/liter to about 1500 kcal/liter and in a further embodiment about 800 kcal/liter to about 1200 kcal/liter. The viscosity of the gelled nutritional composition in one embodiment is about 5000 cp to about 40000 cp. In a further embodiment, the viscosity of the gelled nutritional is typically greater than about 2000 cp (“cp” is centipoise).

In another aspect, this invention provides a method of providing nutrition to a patient suffering from dysphagia, the method comprising administering to the patient a gelled nutritional composition which contains a protein source including gelled whey protein; a carbohydrate source; minerals; and vitamins.

In a further aspect, this invention provides a method for providing supplemental nutrition to a child, the method comprising administering to the child a gelled nutritional composition which contains a protein source including gelled whey protein; a carbohydrate source; minerals; and vitamins.

### DETAILED DESCRIPTION OF THE INVENTION

Definitions:

As used in this application, the following terms have the meanings described below:

a) Viscosity is the ratio of shear stress to shear rate, expressed as dynes-second/cm², or poise. A centripoise is one hundredth of a poise. A poise is a unit of coefficient of viscosity, defined as the tangential force per unit area required to maintain one unit difference in velocity between two parallel planes separated by one centimeter of fluid.Any viscosity determination should be carried out using a Brookfield (model DVII+) viscometer with a #4 spindle. The viscosity is measured by operating the viscometer at a spindle speed that is the highest speed possible to obtain a reading that is on scale.

b) Any reference to a numerical range in this application should be construed as an express disclosure of every number specifically contained within that range and of every subset of numbers contained within that range. Further, this range should be construed as providing support for a claim directed to any number, or subset of numbers in that range. For example, a disclosure of 1-10 should be construed as supporting a range of 2-8, 3-7, 5, 6, 1-9, 3.6-4.6, 3.5-9.9, 1.1-9.9, etc.

Embodiments of the invention are now described by way of example only. This invention provides a nutritional composition which is in the form of a gel. The composition contains a protein source which includes gelled whey protein; a carbohydrate source; minerals; and vitamins. The nutritional composition is ideally suited as a nutritional supplement; especially for children and dysphagia patients.

The protein source includes whey protein. The whey protein may be any commercially available whey protein source which is in substantially intact form. Preferably, however, the whey protein is a whey protein isolate. Whey protein isolates contain more than 90% by weight of whey protein. Suitable whey protein isolate sources include ALACEN 895 (New Zealand Milk Products Inc), BiPRO (Le Sueur Isolates of Le Sueur, Minn.), PROVON-190 (Avonmore Ingredients Inc of Monroe, Wis.) and LACPRODAN 9224 (Royal Proteins, Inc of Rosemone, Ill.).

The protein source may, if desired, included minor amounts of other suitable types of protein. For example, the protein source may further include minor amounts of casein protein, soy protein, rice protein, pea protein, carob protein, oat protein, caseino-glyco-macropeptide or mixtures of these proteins. Further, if desired, the protein sources may further include minor amounts of free amino acids. The other suitable types of protein preferably comprise less than about 20% by weight of the protein sources; more preferably less than about 10% by weight. Most other types of protein do not provide a clear gel and hence the protein source preferably contains little or no protein other than whey protein.

The protein source preferably provides about 5% to about 40% of the total calories of the nutritional composition. More preferably, the protein source provides about 10% and about 25% of the total calories of the nutritional composition; for example about 12% to about 18% and in a further embodiment about 14% to 16%. In terms of weight, the protein preferably provides about 1% to about 5% by weight of the nutritional composition.

The nutritional composition also includes a carbohydrate source. The carbohydrate source preferably provides about 60% to about 95% of the total calories of the nutritional composition. More preferably, the carbohydrate source provides about 75% to about 90% of the total calories of the composition; for example about 80% to about 88%, and in a further embodiment about 84% to about 86%. In terms of weight, the carbohydrate preferably provides about 10% to about 25% by weight of the nutritional composition.

Any suitable carbohydrate source may be used including maltodextrin, starch including modified starch, glucose, fructose, corn syrup and sucrose, or mixtures thereof. Preferably the carbohydrate source is free from lactose. If the nutritional composition contains fruit juices, the carbohydrates in the fruit juice form part of the carbohydrate source.

A preferred carbohydrate source is a mixture of maltodextrin and sucrose or high fructose corn syrup, or both. The maltodextrin preferably has a dextrose equivalent (DE) of at least about 10 but maltodextrins having a DE below 10 are also appropriate. Preferably, the DE of the maltodextrin is about 15. The carbohydrate source preferably comprises about 20% to about 50% by weight of maltodextrin and about 50% to about 80% by weight of sucrose or high fructose corn syrup, or both.

The nutritional composition may include a lipid source but preferably does not. The addition of a lipid source to the nutritional composition will result in a gel which is not totally clear. However, if this is acceptable, a lipid source may be added. If a lipid source is added, it preferably provides about 1% to about 50% of the total calories of the nutritional composition; more preferably about 1% to about 10%. Suitable lipid sources include vegetable oils such as safflower oil, corn oil, canola oil, soybean oil, olive oil, fish oil or other sources of docasahexanic acid and eicosapentanac acid.

The nutritional composition preferably includes a substantially complete vitamin and mineral profile. For example, sufficient vitamins and minerals may be provided to supply about 30% to about 250% of the US recommended daily allowance of the vitamins and minerals per 1000 calories of the nutritional composition; more preferably about 30% to about 100%. Suitable ranges for vitamins and minerals are as follows:

TABLE 1

| Nutrient | Range per 100 g | Alternative range per 100 g |
|---|---|---|
| Sodium | 10 to 50 mg | 25 to 35 mg |
| Potassium | 10 to 50 mg | 25 to 35 mg |
| Calcium | 20 to 270 mg | 40 to 60 mg |
| Magnesium | 0 to 108 mg | 10 to 30 mg |
| Phosphorus | 20 to 270 mg | 85 to 105 mg |
| Iron | 0 to 4.9 mg | 0.5 to 1.0 mg |
| Copper | 0 to 0.54 mg | 0.05 to 0.20 mg |
| Zinc | 0 to 4 mg | 0.80 to 1.10 mg |
| Manganese | 0 to 500 μg | 100 to 200 μg |
| Chromium | 0 to 32.4 μg | 1 to 2.5 μg |
| Molybdenum | 0 to 20.25 μg | 2 to 10 μg |
| Selenium | 0 to 18.9 μg | 1 to 5 μg |
| Iodine | 0 to 41 μg | 2 to 10 μg |
| Vitamin C | 0 to 16.2 mg | 5 to 15 mg |
| Vitamin B1 | 0 to 0.4 mg | 0.05 to 0.2 mg |
| Vitamin B2 | 0 to 0.46 mg | 0.05 to to 0.2 mg |
| Niacin | 0 to 5.4 mg | 1 to 2 mg |
| Vitamin B6 | 0 to 0.54 mg | 0.1 to 0.3 mg |
| Vitamin B12 | 0 to 1.62 μg | 0.2 to 0.6 μg |
| Pantothenic acid | 0 to 2.7 | 0.5 to 1 mg |
| Folic acid | 0 to 108 μg | 10 to 50 μg |
| Biotin | 0 to 81 μg | 10 to 30 μg |
| Vitamin A | 0 to 331.8 IU | 150 to 300 IU |
| β-carotene | 0 to 0.1 mg | 0.05 to 0.09 mg |
| Vitamin D3 | 0 to 28.06 IU | 10 to 25 IU |
| Vitamin E | 0 to 2.31 IU | 1 to 2 IU |
| Vitamin K | 0 to 4.01 μg | 2 to 3 μg |
| Choline | 0 to 26.1 mg | 20 to 25 mg |
| Taurine | 0 to 4.99 mg | 4 to 4.5 mg |

In a further embodiment, Table 2 depicts a typical nutrient profile for one of the gels of this invention:

TABLE 2

| Nutrient | Range per 100 g |
|---|---|
| Sodium, mg | 84.46 to 270.27 |
| Potassium, mg | 118.24 to 394.14 |
| Calcium, mg | 33.78 to 112.61 |
| Magnesium, mg | 13.51 to 45.05 |
| Phosphorus, mg | 33.78 to 112.61 |
| Iron, mg | 0.61 to 2.03 |
| Copper, mg | 0.07 to 0.23 |
| Zinc, mg | 0.51 to 1.69 |
| Manganese, mg | 0.07 to 0.23 |
| Chromium, mcg | 4.05 to 13.51 |
| Molybdenum, mcg | 2.53 to 8.45 |
| Selenium, mcg | 2.36 to 7.88 |
| Iodine, mcg | 5.07 to 16.89 |
| Vitamin C, mg | 2.03 to 6.76 |
| Vitamin B1, mg | 0.05 to 0.17 |
| Vitamin B2, mg | 0.06 to 0.19 |
| Niacin, mg | 0.68 to 2.25 |
| Vitamin B6, mg | 0.07 to 0.23 |
| Vitamin B12, mcg | 0.20 to 0.68 |
| Pantothenic acid, mg | 0.34 to 1.13 |
| Folic acid, mcg | 13.51 to 45.05 |
| Biotin, mcg | 10.14 to 33.78 |
| Vitamin A, IU | 168.92 to 563.06 |
| β-carotene, mg | 0.00 to 0.10 |
| Vitamin D3, IU | 13.51 to 45.05 |
| Vitamin E, IU | 1.01 to 3.38 |
| Vitamin K, mcg | 2.70 to 9.01 |
| Choline, mg | 0.00 to 26.10 |
| Taurine, mg | 0.00 to 4.99 |

The nutritional composition typically has a energy content of about 800 kcal/liter to about 1200 kcal/liter; for example an energy content of about 1000 kcal/liter. One liter of the gelled composition will typically weigh from about 1000 to about 1400 grams, and in a further embodiment about 1100 to about 1200 grams.

The nutritional composition preferably has a pH in the range of about 2.0 to about 4.5, and in a further embodiment about 2.8 to about 3.3, more typically from about 3.5 to about 4.1. Therefore food grade acidifying agents are preferably included. Examples of suitable acidifying agents include food grade citric, acid, malic acid, hydrochloric acid, lactic acid, phosphoric acid, or mixtures of these acids. Mixtures of malic and phosphoric and citric and phosphoric are suitable. If a fruit juice is included in the nutritional composition, the fruit juice will also provide some acidifying agents.

The nutritional composition may include flavors as desired. If a fruit juice is included in the nutritional composition, the fruit juice will provide flavor. However, artificial flavors may also be used; either alone or in addition to natural flavors.

The nutritional composition typically has a viscosity above about 2000 centipoise, more typically above about 5000 centipoise (“cp”); more preferably about 8000 cp to about 40000 cp. If a soft gel is desired, viscosities in the range of about 10000 cp to about 15000 are suitable. For pudding-like products, viscosities in the range of about 30000 cp to about 38000 cp are desirable.

The nutritional composition may be produced by suspending the protein source in water under stirring. The carbohydrate source, the lipid source if used, the vitamins and minerals, acidifying agents, flavors and colors may be added to provide a liquid mixture. Commercially available liquefiers may be used to form the liquid mixture. The temperature of the liquid mixture may be room temperature but high temperatures may also be used if desired.

The liquid mixture is then standardized to provide a desired solids content; for example of about 20% to about 30% by weight. More preferably, the solids content is about 24% to about 26%. The pH may be standardized to about 2.0 to about 4.5.

If the liquid mixture contains lipids, the mixture may then be homogenized; for example in two stages at about 7 MPa to about 40 MPa in the first stage and about 2 MPa to about 14 MPa in the second stage. If not done prior to homogenization, the pH and solids content of the homogenized mixture may be standardized at this point. Homogenization is not necessary for products which do not contain lipids.

The standardized (optionally homogenized) mixture is then filled into containers and gelled. This may be accomplished in many ways. For example, the standardized mixture may be rapidly heated to about 90 to 95° C. in a plate heat exchanger and then hot filled into containers. The containers are sealed and held at a temperature of at least about 80° C. for a time sufficient to complete gelled of the whey protein; for example for about 5 to 10 minutes. The containers are then cooled to ambient temperature to provide a gelled nutritional composition.

Alternatively, the standardized mixture may be filled into containers at ambient temperature. The containers are sealed and then heated to a temperature of at least about 80° C. for a time sufficient to gel the whey protein; for example for about 10 to 20 minutes. The containers are then cooled to ambient temperature to provide a gelled nutritional composition.

The nutritional compositions may be used as nutritional supplements for dysphagia patients. The amount of the nutritional composition required to be consumed by a patient will vary depending upon factors such as the patient's condition, the patient's body weight, the age of the patient, the other sources of nutrition. However, the required amount may be readily set by a medical practitioner or nutritionist.

Similarly, the nutritional composition may be utilized to provide nutritional support post operatively for surgery patients. The protein, vitamins, and calories will promote the patients recovery. The amount of the nutritional composition required for the post op patient will vary depending upon factors such as the patient's condition, the patient's body weight, the age of the patient, the other sources of nutrition. However, the required amount may be readily set by a medical practitioner or nutritionist.

Similarly, the nutritional compositions may be used as nutritional supplements in general; especially for children. The amount of the nutritional composition required to be consumed is not critical.

The nutritional compositions may also be used as nutritional supplements for athletes. In this for, the nutritional compositions are preferably packaged into squeeze packs which are easy to handle and use when active.

Specific examples of the invention are now described by way of further illustration.

### EXAMPLE 1

Water at room temperature is placed in a batch mixing tank and stirred at a moderate speed. Whey protein isolate (ALACEN 895) is added to the water. A carbohydrate mixture of maltodextric (DE 15), a sweetener and optionally a starch is added to solution under stirring. Minerals are then added. The solution is then acidified to a pH of about 3.6 using an acid mixture of malic acid and phosphoric acid and fruit juices. Flavors, color and vitamins are then added. The solids content of the solution is then standardized to the desired level if necessary.

The solution is then rapidly heated to about 90 to 95° C. in a plate heat exchanger and hot filled into containers. The containers are sealed and held at a temperature of at least about 80° C. for about 5 to 10 minutes. The products are then cooled to ambient temperature. The composition of the products is an follows:

TABLE 3

| Nutrient | Concentration (weight %) |
|---|---|
| Whey protein isolate | 2-4.5 |
| Carbohydrates | 11-20 |
| Maltodextrin | 5-8 |
| Starch | 0-1 |
| Sucrose and/or high fructose corn | 6-11 |
| syrup. | |
| Fruit Juice | 5-50 |

The vitamin and mineral profile of the products is as follows:

TABLE 4

| | Amount per |
|---|---|
| Nutrient | 100 g |
| Sodium | 28 mg |
| Potassium | 28 mg |
| Calcium | 50 mg |
| Magnesium | 20 mg |
| Phosphorus | 94 mg |
| Iron | 0.84 mg |
| Copper | 0.10 mg |
| Zinc | 0.93 mg |
| Manganese | 141 μg |
| Chromium | 1.7 μg |
| Molybdenum | 6.2 μg |
| Selenium | 3.1 μg |
| iodine | 6.75 μg |
| Vitamin C | 11.25 mg |
| Vitamin B1 | 0.11 mg |
| Vitamin B2 | 0.14 mg |
| Niacin | 1.46 mg |
| Vitamin B6 | 0.2 mg |
| Vitamin B12 | 0.42 μg |
| Pantothenic acid | 0.74 |
| Folic acid | 28.14 μg |
| Biotin | 22 μg |
| Vitamin A | 225 IU |
| β-carotene | 0.07 mg |
| Vitamin D3 | 18.75 IU |
| Vitamin E | 1.52 IU |
| Vitamin K | 2.64 μg |
| Choline | 23.92 mg |
| Taurine | 4.22 mg |

The products have solids concentration of about 24 to about 26% by weight and a pH of about 3.5 to about 4.1. The products are the form of a clear gel. The energy density of the products is 1000 kcal/1. The viscosities range from about 10000 cp to about 35000 cp when measured using a Brookfield LV-1 viscometer with a No 4 spindle at 12 rpm.

### EXAMPLE 2

The process of example 1 is repeated except that the standardized solution is filled into containers at ambient temperature. The containers are sealed and held at a temperature of at least about 80° C. for about 10 to 20 minutes. The products are then cooled to ambient temperature.

The products are standardized to a solids concentration of about 24 to about 26% by weight and to a pH of about 3.5 to about 4.1. The products are in the form of clear gel. The energy density of the products is 1000 kcal/1. The viscosities range from about 10000 cp to about 35000 cp when measured using a Brookfield LV-1 viscometer with a No 4 spindle at 12 rpm.

### EXAMPLE 3

A nutritional composition is produced according to example 1 to produce a viscosity of about 35000. This product may be given to an elderly patient suffering from a swallowing disorder. The patient is able to consume the nutritional supplement without great difficulty and much easier than liquids.

### EXAMPLE 4

Utilizing the procedure of Example 1, a gelled composition was prepared having the composition outlined below in Table 5.

TABLE 5

| | Amount |
|---|---|
| | Kg/1000 Kg |
| Ingredients | Apple Flavor |
| Water | ˜703.8* |
| Maltrin 200 | 159.64 |
| Sucrose | 81.67 |
| Whey Protein Isolate | 40.6 |
| Citric Acid | 7.05 |
| Phosphoric Acid 75% | 2.36 |
| Calcium Chloride | 2.79 |
| Apple Flavor | .7 |
| Asorbic Acid | .5351 |
| Caramel Liquid Color | .4 |
| Ultra Trac Minerals/Trace | .2205 |
| Minerals Premix | |
| Vitamin ADEK Premix | .1784 |
| Vitamin Premix | .0379 |
| Folic Acid | .0013 |
| Potassium Iodide | .0002042 |

*Water weight is an approximation and is based on a final total solids of 28.6 g/100 g and a blending solids of 35.5 g/100 g

### EXAMPLE 5

Utilizing the procedure of Example 1, a gelled composition was parepared having the composition outlined below in Table 6.

TABLE 6

| | Amount |
|---|---|
| | Kg/1000 Kg |
| Ingredients | Apple Flavor |
| Water | ˜703.8* |
| Maltrin 200 | 188.6 |
| Sucrose | 60.3 |
| Whey Protein Isolate Aria | 42.0 |
| (MD) 9224 | |
| Citric Acid | 2.71 |
| Phosphoric Acid 75% | 0 |
| Calcium Chloride | 0 |
| Apple Flavor | .7 |
| Asorbic Acid | .5351 |
| Caramel Liquid Color | .4 |
| UTM/TM Premix | .1373 |
| Vitamin ADEK Premix | 0 |
| Water Soluble | |
| Vitamin Premix Med | .0379 |
| Nutrition | |
| Folic Acid | 0 |
| Potassium Iodide | 0 |
| HCl (37%) | 2.91 |

*Water weight is an approximation and is based on a final total solids of 28.6 g/100 g and a blending solids of 35.5 g/100 g

We claim:

1. A gelled nutritional composition comprising a protein source including gelled whey protein; a carbohydrate source; minerals; and vitamins.

2. The gelled nutritional composition according to claim 1 which is a clear gel.

3. The gelled composition according to claim 1 which has a pH of from about 2.0 to about 4.5.

4. The gelled composition according to claim 1 having a viscosity of at least about 2000 centipoise.

5. The gelled composition according to claim 1 having an energy density of at least 600 kcal per liter.

6. The gelled nutritional composition according to claim 1 in which provides about 30% to about 100% of the recommended daily allowance of vitamins and minerals per 1000 calories.

7. The gelled nutritional composition according to claim 1 in which has an energy density of about 800 kcal/lter to about 1500 kcal/liter.

8. The gelled nutritional composition according to claim 1 which has a viscosity of about 5000 cp to about 40000 cp.

9. The gelled nutritional composition according to claim 1 in which the protein source provides about 10% to about 25% of the total calories of the composition.

10. The gelled nutritional composition according to claim 1 in which the carbohydrate source provides about 75% to about 90% of the total calories of the composition.

11. A gelled nutritional in the form of a clear gel, the composition comprising a protein source including a gelled whey protein; a carbohydrate source; minerals; and vitamins.

12. The gelled nutritional composition according to claim 11 which provides about 30% to about 100% of the recommended daily allowance of vitamins and minerals per 1000 calories.

13. The gelled nutritional composition according to claim 11 which has an energy density of about 800 kcal/liter to about 1200 kcal/liter.

14. The gelled nutritional composition according to claim 11 which has a viscosity of about 5000 cp to about 40000 cp.

15. The gelled nutritional composition according to claim 11 in which the protein source provides about 10% to about 25% of the total calories of the composition.

16. The gelled nutritional composition according to claim 11 in which the carbohydrate source provides about 75% to about 90% of the totalcalories of the composition.

17. The gelled nutritional composition according to claim 11 which has a pH of about 3.5 to about 4.1.

18. A gelled nutritional composition in the form of a clear gel, the composition comprising a protein source including gelled whey protein; a carbohydrate source; minerals; and vitamines; and theocmposition having a viscosity of about 5000 cp to about 40000 cp.

19. A method of providing nutrition to a patient suffering from dysphagia, the method comprising administering to the patient to gelled nutritional composition which contains a protein source including gelled whey protein; a carbohydrate source; minerals; and vitamins.

20. A method for providing supplemental nutrition to a child the method comprising administering asaid patient the gelled composition according to claim 1.

21. A method of providing nutrition to a patient after surgery, the method comprising administering a said patient the gelled composition according to claim 1.
